(19) United States
(12) Patent Application Publication
(10) Pub. No.: US 2021/0103138 A1
(43) Pub. Date: Apr. 8, 2021

(54) ILLUMINATION OPTICAL SYSTEM AND ENDOSCOPE SYSTEM

(71) Applicant: OLYMPUS CORPORATION, Tokyo (JP)

(72) Inventor: Kazunari HANANO, Tokyo (JP)

(73) Assignee: OLYMPUS CORPORATION, Tokyo (JP)

(21) Appl. No.: 17/124,534

(22) Filed: Dec. 17, 2020

Related U.S. Application Data
(63) Continuation of application No. PCT/JP2018/023793, filed on Jun. 22, 2018.

(51) Int. Cl. G02B 23/24

(52) U.S. Cl. CPC G02B 23/2469; A61B 1/07; G02B 23/243

(57) ABSTRACT

An illumination optical system includes: a light source; a light guide member that includes an input end and a plurality of output ends, optically guides light entering the input end, and outputs the light from the plurality of output ends; and a deflecting element that deflects the light from the light source toward the input end and changes a position where the light enters an end surface of the input end. The end surface of the input end is divided into a plurality of areas. Light entering one of the areas is output from one of the output ends different from an output end from which light entering another one of the areas is output.

### CROSS-REFERENCE TO RELATED APPLICATIONS

This is a continuation of International Application PCT/JP2018/023793 which is hereby incorporated by reference herein in its entirety.

### TECHNICAL FIELD

The present invention relates to illumination optical systems and endoscope systems.

### BACKGROUND ART

A conventional illumination optical system for an endoscope is equipped with a light source, an optical fiber, and an illumination lens provided at the distal end of a scope (e.g., see Patent Literatures 1 to 5). Light output from the light source is optically guided by the optical fiber and is radiated onto a subject from the illumination lens.

### CITATION LIST

### Patent Literature

### {PTL 1}

### The Publication of Japanese Patent No. 458843

### {PTL 2}

### Japanese Unexamined Patent Application, Publication No. 2002-98913

### {PTL 3}

### Japanese Unexamined Patent Application, Publication No. 2016-2302

### {PTL 4}

### Japanese Unexamined Patent Application, Publication No. 2010-243874

### {PTL 5}

### Japanese Unexamined Patent Application, Publication No. 2005-328990

### SUMMARY OF INVENTION

An aspect of the present invention provides an illumination optical system including: a light source; a light guide member that includes an input end and a plurality of output ends, optically guides light entering the input end, and outputs the light from the plurality of output ends; and a deflecting element that deflects the light from the light source toward the input end and that changes a position where the deflected light enters an end surface of the input end. The end surface of the input end is divided into a plurality of areas. Light entering one of the plurality of areas is output from one of the output ends different from an output end from which light entering another one of the areas is output.

### BRIEF DESCRIPTION OF DRAWINGS

FIG. 1 illustrates the overall configuration of an endoscope system according to an embodiment of the present invention.

FIG. 2 is a perspective view of a distal end of a scope in the endoscope system in FIG. 1.

FIG. 3A illustrates the overall configuration of an illumination optical system in the endoscope system in FIG. 1.

FIG. 3B illustrates shifting of illumination light caused by rotating a galvanometer mirror in the illumination optical system in FIG. 3A.

FIG. 4 is a front view of an end surface of an input end of a light guide member and illustrates an example of a plurality of areas.

### DESCRIPTION OF EMBODIMENTS

An illumination optical system 1 and an endoscope system 100 according to an embodiment of the present invention will be described below with reference to the drawings.

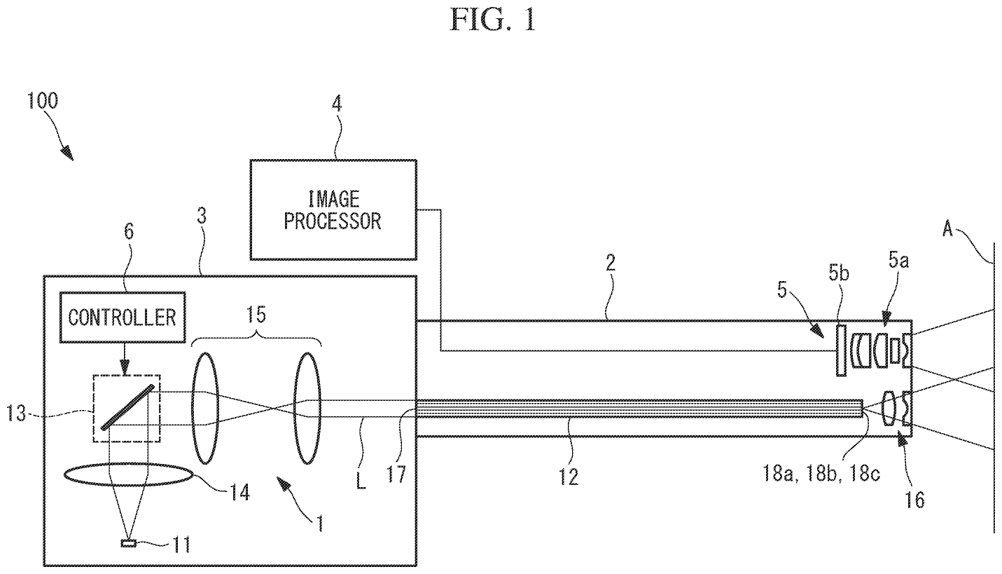

As shown in FIG. 1, the endoscope system 100 according to this embodiment includes a long scope 2, a light source device 3 connected to the base end of the scope 2, and an image processor 4. The endoscope system 100 also includes an imaging optical system 5 that acquires an image of a subject A, the illumination optical system 1 that illuminates the field of view of the imaging optical system 5, and a controller 6 that controls the illumination optical system 1.

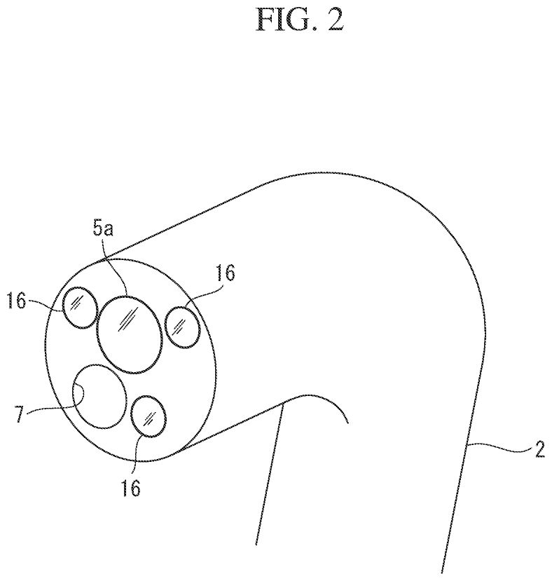

The imaging optical system 5 includes an imaging lens 5a and an image sensor 5b. As shown in FIG. 2, the imaging lens 5a is disposed at the distal-end surface of the scope 2 and forms an image of light from the subject A. The image sensor 5b is disposed within the scope 2 and generates an image signal by acquiring the image of the subject A formed by the imaging lens 5a. The image signal is transmitted from the image sensor 5b to the image processor 4. The image processor 4 generates an image from the image signal and causes a display device (not shown) to display the image. Reference sign 7 denotes a channel in which, for example, a surgical device is fitted.

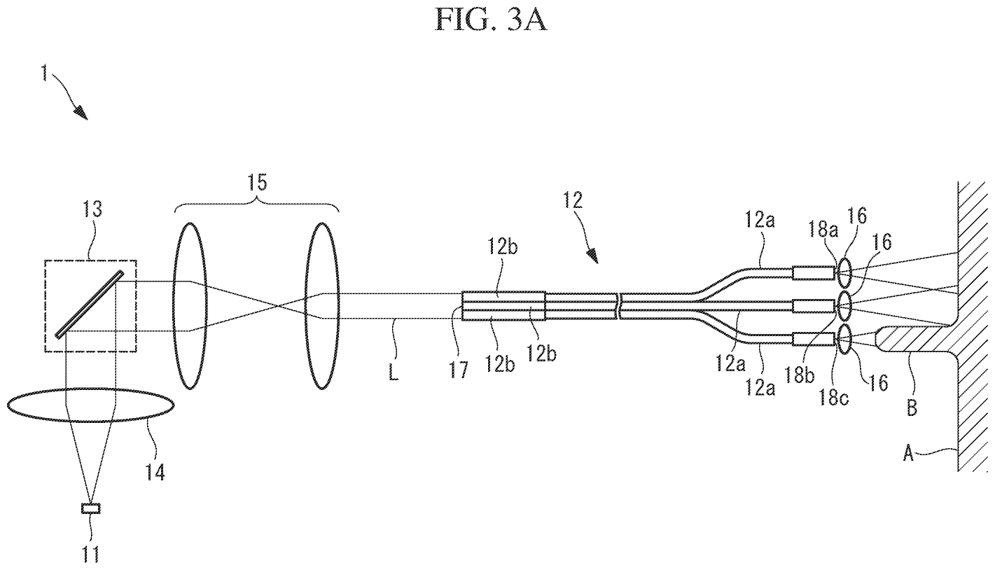

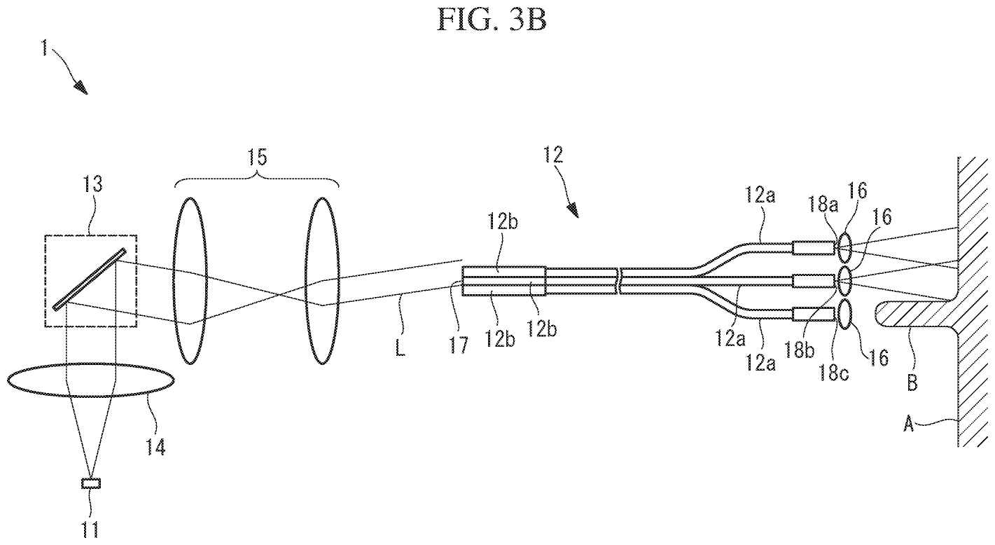

As shown in FIGS. 3A and 3B, the illumination optical system 1 includes a light source 11 that emits illumination light L, a long light guide member 12 that optically guides the illumination light L, a galvanometer mirror (deflecting element) 13 that deflects the illumination light L from the light source 11 toward an input end 17 of the light guide member 12, a first lens group 14 disposed between the light source 11 and the galvanometer mirror 13, a second lens group 15 disposed between the galvanometer mirror 13 and the light guide member 12, and a plurality of illumination lenses 16 that output the illumination light L optically guided by the light guide member 12 toward the subject A.

The light source 11, the galvanometer mirror 13, the first lens group 14, and the second lens group 15 are disposed within the light source device 3. The light guide member 12 is disposed in the longitudinal direction within the scope 2 from the base end to near the distal end of the scope 2. As shown in FIG. 2, the illumination lenses 16 are disposed at the distal-end surface of the scope 2.

The light source 11 is a solid-state light source, such as an LED (light-emitting diode).

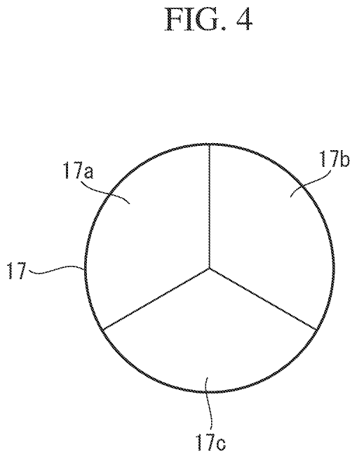

The light guide member 12 has a single input end 17 at the base end and a plurality of output ends 18a, 18b, and 18c at the distal end. The output ends 18a, 18b, and 18c are disposed at positions different from one another in a direction intersecting the longitudinal direction of the light guide member 12. The light guide member 12 optically guides the illumination light L from the input end 17 to the output ends 18a, 18b, and 18c and outputs the illumination light L from the output ends 18a, 18b, and 18c. As shown in FIG. 4, the end surface of the input end 17 is divided into a plurality of areas 17a, 17b, and 17c. The number of areas 17a, 17b, and 17c is equal to the number of output ends 18a, 18b, and 18c. In the reference drawings, three areas 17a, 17b, and 17c and three output ends 18a, 18b, and 18c are provided.

In FIG. 4, the end surface of the input end 17 is equally divided in the circumferential direction around the center such that fan-shaped areas 17a, 17b, and 17c are arranged in the circumferential direction. However, the pattern in which the end surface of the input end 17 is divided is not limited to this. For example, the end surface of the input end 17 may be divided into a plurality of concentric ring-shaped areas. As another alternative, the end surface of the input end 17 may be divided into a plurality of areas arranged in a single row.

The areas 17a, 17b, and 17c and the output ends 18a, 18b, and 18c have a one-to-one correspondence relationship. Specifically, the area 17a corresponds to the output end 18a, the area 17b corresponds to the output end 18b, and the area 17c corresponds to the output end 18c. An optical path from the area 17a to the output end 18a, an optical path from the area 17b to the output end 18b, and an optical path from the area 17c to the output end 18c are independent from one another. Therefore, a light beam entering one area is output from one output end that is different from the two output ends from which light beams entering the two remaining areas are output. The illumination lenses 16 are disposed at positions facing the output ends 18a, 18b, and 18c.

Such a light guide member 12 is constituted of, for example, a plurality of optical fiber bundles 12a. Each optical fiber bundle 12a is constituted of a plurality of narrow optical fibers bound together into a single bundle. The base ends of the plurality of optical fiber bundles 12a are bound together. For example, the base ends of the plurality of optical fiber bundles 12a are accommodated within a tubular frame the interior of which is partitioned into a plurality of spaces. The illumination light L emitted by the light source 11 normally has an intensity distribution in which the intensity decreases from the center toward the periphery. In order to make the intensity of the illumination light L entering the optical fiber bundles 12a uniform, the base end of each optical fiber bundle 12a may be connected to a light guide rod 12b having a light scattering function.

The galvanometer mirror 13 is disposed on the optical axis of the illumination light L between the light source 11 and the galvanometer mirror 13 and is rotatable about a rotation axis that is orthogonal to the optical axis. The galvanometer mirror 13 deflects the illumination light L from the light source 11 in a direction parallel to or substantially parallel to the optical axis of the light guide member 12. As shown in FIG. 3B, when the galvanometer mirror 13 is rotated, the illumination light L entering the input end 17 of the light guide member 12 via the second lens group 15 shifts in a direction intersecting the optical axis between the galvanometer mirror 13 and the input end 17, so that the position where the illumination light L enters the end surface of the input end 17 changes, whereby the amount of illumination light L entering each of the areas 17a, 17b, and 17c changes. Consequently, the amounts of illumination light L output from the plurality of output ends 18a, 18b, and 18c can be changed independently from one another in accordance with the rotational angle of the galvanometer mirror 13. The galvanometer mirror 13 may be a two-axis galvanometer mirror rotatable around two rotation axes that intersect each other such that the position where the illumination light L enters the end surface of the input end 17 can be changed two-dimensionally.

The first lens group 14 includes at least one lens. The first lens group 14 uses the at least one lens to substantially collimate the illumination light L in the form of divergent light emitted by the light source 11, and outputs the substantially collimated light toward the galvanometer mirror 13.

The second lens group 15 includes at least one lens. The second lens group 15 guides the illumination light L, the deflection direction of which changes by rotating the galvanometer mirror 13, to the input end 17. In the reference drawings, the second lens group 15 includes a pair of lenses. The lens at the galvanometer mirror 13 side receives the illumination light L deflected by the galvanometer mirror 13, whereas the lens at the light guide member 12 side substantially collimates the illumination light L. With such a second lens group 15, the beam diameter of the illumination light L is adjusted to a dimension suitable for the size of the end surface of the input end 17. Furthermore, the illumination light L enters the input end 17 from the second lens group 15 in a direction parallel to the optical axis of the light guide member 12, regardless of the rotational angle of the galvanometer mirror 13.

The controller 6 controls the rotational angle of the galvanometer mirror 13 based on a user command. The user command is input to the controller 6 by using, for example, an input device (not shown) connected to the controller 6.

Next, the operation of the illumination optical system 1 and the endoscope system 100 having the above-described configuration will be described.

In the endoscope system 100 according to this embodiment, the illumination light L in the form of divergent light emitted by the light source 11 is substantially collimated by the first lens group 14, is deflected by the galvanometer mirror 13, and is guided to the input end 17 of the light guide member 12 by the second lens group 15. The illumination light L entering the end surface of the input end 17 is output from the plurality of output ends 18a, 18b, and 18c and is radiated onto the subject A from the plurality of illumination lenses 16. The illumination light L output from the plurality of illumination lenses 16 illuminates different regions of the subject A.

The illumination light L reflected at the subject A is received by the imaging lens 5a. An image of the subject A formed by the imaging lens 5a is acquired by the image sensor 5b, and an image signal is transmitted from the image sensor 5b to the image processor 4. Then, the image processor 4 generates the image of the subject A from the image signal, and the image is displayed on the display device.

A user determines whether or not the subject A within the field of view of the imaging optical system 5 is appropriately illuminated with the illumination light L based on the image displayed on the display device. If halation is occurring in the image, the user inputs a command for rotating the galvanometer mirror 13 to the controller 6 so as to change the rotational angle of the galvanometer mirror 13 in a direction for reducing the amount of illumination light L entering the area 17a, 17b, or 17c corresponding to the region in which the halation is occurring.

For example, as shown in FIG. 3A, if the subject A has an irregular surface, the distance from the output ends 18a, 18b, and 18c to the surface of the subject A varies. In the example in FIG. 3A, a protrusion B, such as a plica within the intestine, is located near the output end 18c. Therefore, the protrusion B is illuminated with intense illumination light L, thus causing halation to occur in the region of the protrusion B in the image. The user rotates the galvanometer mirror 13 downward in FIG. 3B to move the position where the illumination light L enters the end surface of the input end 17 upward, thereby reducing the amount of illumination light L entering the area 17c. Consequently, the amount of illumination light L output from the output end 18c can be selectively reduced, so that the halation in the image can be eliminated. In this case, the amount of illumination light L from the remaining output ends 18a and 18b is maintained, so that the brightness in the remaining regions within the image is maintained.

Accordingly, in this embodiment, different regions in the field of view of the imaging optical system 5 are illuminated with the illumination light L from the plurality of output ends 18a, 18b, and 18c. Furthermore, the end surface of the input end 17 is divided into the plurality of areas 17a, 17b, and 17c, and the areas 17a, 17b, and 17c correspond to the output ends 18a, 18b, and 18c that are different from one another. Therefore, by using the galvanometer mirror 13 to change the position where the illumination light L enters the end surface of the input end 17, the amounts of illumination light L output from the output ends 18a, 18b, and 18c are adjusted independently of one another, so that the brightness in the field of view can be partially and dynamically adjusted during observation of the subject A. This is advantageous in that the subject A can be illuminated with the illumination light L appropriate for the scene, so that halation can be reduced.

As an alternative to this embodiment in which the deflecting element is the galvanometer mirror 13, the deflecting element may be a MEMS (microelectromechanical system) mirror device.

The MEMS mirror device has a plurality of micromirrors arranged in a plane, and the angle of each micromirror is adjustable. Similar to the galvanometer mirror 13, the position where the illumination light L enters the end surface of the input end 17 can be changed by changing the angle of each micromirror.

The MEMS mirror device may be a DMD (digital micromirror device, registered trademark).

The DMD can apply a freely-chosen light distribution pattern to the cross section of the beam of the illumination light L entering the DMD by individually controlling the angles of the micromirrors. In detail, the DMD selectively switches the angle of each micromirror between two angles so as to switch the light reflected by each micromirror between on-light and off-light. On-light is output from the DMD to the second lens group 15. Off-light is not output outside the DMD.

The DMD is disposed at a position optically conjugate with the input end 17, and a light distribution pattern identical to the light distribution pattern of the illumination light L formed by the DMD is formed on the end surface of the input end 17. Therefore, without rotating, the DMD changes the position where the illumination light L enters the end surface of the input end 17 by switching the angle of each micromirror.

In this embodiment, the controller 6 may control the galvanometer mirror 13 based on the image of the subject A.

For example, the controller 6 detects halation in the image based on a pixel value and changes the rotational angle of the galvanometer mirror 13 in the direction for reducing the amount of illumination light L entering the area 17a, 17b, or 17c corresponding to the region where the halation is occurring. Consequently, the halation in the image can be automatically eliminated.

In this embodiment, a distance measuring unit that measures the distance between the plurality of output ends 18a, 18b, and 18c and the subject A may be provided. The controller 6 may control the galvanometer mirror 13 based on the distance measured by the distance measuring unit.

For example, if the light source 11 is a laser light source, the distance measuring unit may be a TOF (time of flight) distance measuring sensor. The distance measuring unit may be a structured-illumination distance measuring sensor that projects, for example, a lattice, striped, or random-dot-type light-and-dark pattern onto the subject A.

The TOF distance measuring sensor includes a light detector with high time resolution and has a function for calculating a distance from a signal obtained by the light detector. The structured-illumination distance measuring sensor has a function for projecting a light-and-dark pattern and calculating a distance from an acquired image. In either type, the controller 6 changes the rotational angle of the galvanometer mirror 13 in the direction for reducing the amount of illumination light L output from the output end 18a, 18b, or 18c located at the closest distance to the subject A. Consequently, the light distribution characteristics of the illumination light L output from the output end 18a, 18b, or 18c can be appropriately controlled in accordance with the measured distance, so that the quality of the image can be improved.

The above-described embodiment leads to the following aspects.

An aspect of the present invention provides an illumination optical system including: a light source; a light guide member that includes an input end and a plurality of output ends, optically guides light entering the input end, and outputs the light from the plurality of output ends; and a deflecting element that deflects the light from the light source toward the input end and that changes a position where the deflected light enters an end surface of the input end. The end surface of the input end is divided into a plurality of areas. Light entering one of the plurality of areas is output from one of the output ends different from an output end from which light entering another one of the areas is output.

According to this aspect, the light from the light source is deflected by the deflecting element, enters the input end of the light guide member, and is output from the plurality of output ends of the light guide member so as to illuminate the subject. The end surface of the input end is divided into a plurality of areas, such that the light entering the plurality of areas is output from the different output ends to illuminate different regions of the subject. Therefore, by using the deflecting element to change the position where the light enters the end surface of the input end, the amounts of light illuminating the individual regions of the subject can be adjusted independently of each other. Accordingly, the subject can be illuminated with illumination light appropriate for the scene, so that halation can be reduced.

In the above aspect, the illumination optical system may further include a first lens group that includes at least one lens and that is disposed between the light source and the deflecting element. The first lens group may substantially collimate the light from the light source.

According to this configuration, the light entering the deflecting element and the light deflected by the deflecting element can be controlled to have constant beam diameters.

In the above aspect, the deflecting element may be a galvanometer mirror disposed on an optical axis of the light from the light source.

By rotating the galvanometer mirror, the deflection angle of the light can be changed, so that the position where the light enters the end surface of the input end can be changed.

In the above aspect, the deflecting element may be a MEMS mirror device that includes a plurality of micromirrors and in which an angle of each of the plurality of micromirrors is adjustable.

By changing the angle of each micromirror, the deflection angle of the light can be changed, so that the position where the light enters the end surface of the input end can be changed.

In the above aspect, the illumination optical system may further include a second lens group that includes at least one lens and that is disposed between the deflecting element and the input end. The second lens group may guide the light deflected by the deflecting element to the input end of the light guide member.

With this configuration, the light can be made to enter the input end of the light guide member from the deflecting element, regardless of a change in the angle by which the light is deflected by the deflection element.

In the above aspect, the MEMS mirror device may be a DMD (registered trademark) and may be disposed at a position optically conjugate with the input end of the light guide member.

By using the DMD, the light distribution pattern of the cross section of the beam of the light from the light source can be changed, so that the position where the light enters the end surface of the input end of the light guide member disposed at the position optically conjugate with the DMD can be changed.

Another aspect of the present invention provides an endoscope system including the aforementioned illumination optical system, an imaging optical system that acquires an image of a subject illuminated with the light output from the plurality of output ends of the illumination optical system, and a controller that controls the deflecting element to change the position where the light enters the end surface of the input end.

In the above aspect, the controller may control the deflecting element based on the image of the subject acquired by the imaging optical system.

With this configuration, the deflecting element can be automatically controlled by the controller such that the image has appropriate brightness.

In the above aspect, the endoscope system may further include a distance measuring unit that measures a distance between each of the plurality of output ends and the subject. The controller may control the deflecting element based on the distance measured by the distance measuring unit.

Since the intensity of the illumination light that illuminates the subject increases with decreasing distance between each output end and the subject, halation is more likely to occur. The controller can automatically control the deflecting element such that each region of the subject is irradiated with illumination light having the intensity

### REFERENCE SIGNS LIST

1 illumination optical system
2 scope
3 light source device
4 image processor
5 imaging optical system
6 controller
11 light source
12 light guide member
13 galvanometer mirror (deflecting element)
14 first lens group
15 second lens group
16 illumination lens
17 input end
17a, 17b, 17c area
18a, 18b, 18c output end
100 endoscope system

1. An illumination optical system comprising:
a light source;
a light guide member that comprises an input end and a plurality of output ends, the light guide member optically guiding light entering the input end and outputting the light from the plurality of output ends; and
a deflecting element that deflects the light from the light source toward the input end and that changes a position where the deflected light enters an end surface of the input end,
wherein the end surface of the input end is divided into a plurality of areas, and
wherein light entering one of the plurality of areas is output from one of the output ends different from an output end from which light entering another one of the areas is output.

2. The illumination optical system according to claim 1, further comprising:
a first lens group that comprises at least one lens and that is disposed between the light source and the deflecting element,
wherein the first lens group substantially collimates the light from the light source.

3. The illumination optical system according to claim 1,
wherein the deflecting element is a galvanometer mirror disposed on an optical axis of the light from the light source.

4. The illumination optical system according to claim 1,
wherein the deflecting element is a MEMS mirror device that comprises a plurality of micromirrors and in which an angle of each of the plurality of micromirrors is adjustable.

5. The illumination optical system according to claim 1, further comprising:
a second lens group that comprises at least one lens and that is disposed between the deflecting element and the input end,
wherein the second lens group guides the light deflected by the deflecting element to the input end of the light guide member.

6. The illumination optical system according to claim 4,
wherein the MEMS mirror device is a DMD (registered trademark) and is disposed at a position optically conjugate with the input end of the light guide member.

7. An endoscope system comprising:
the illumination optical system according to claim 1;
an imaging optical system that comprises an image sensor and that is configured to acquire an image of a subject illuminated with the light output from the plurality of output ends of the illumination optical system; and
a controller configured to control the deflecting element to change the position where the light enters the end surface of the input end.

8. The endoscope system according to claim 7,
wherein the controller is configured to control the deflecting element based on the image of the subject acquired by the imaging optical system.

9. The endoscope system according to claim 7, further comprising:
a distance measuring sensor configured to measure a distance between each of the plurality of output ends and the subject,
wherein the controller is configured to control the deflecting element based on the distance measured by the distance measuring sensor.
